(19) United States
(12) Patent Application Publication
(10) Pub. No.: US 2020/0408860 A1
(43) Pub. Date: Dec. 31, 2020

(54) SYSTEMS FOR A RADIO FREQUENCY COIL ASSEMBLY

(71) Applicant: General Electric Company, Schenectady, NY (US)

(72) Inventors: Victor Taracila, Beachwood, OH (US); Mark Giancola, Chesterland, OH (US); Fraser John Laing Robb, Aurora, OH (US); Balint Franko, Streetsboro, OH (US); Clyve Konrad Rosales Follante, Twinsburg, OH (US); Yun Jeong Stickle, Solon, OH (US)

(21) Appl. No.: 16/453,963

(22) Filed: Jun. 26, 2019

(51) Int. Cl. G01R 33/34; G01R 33/3415; A61B 5/055

(52) U.S. Cl. CPC G01R 33/34084; A61B 5/055; G01R 33/3415

(57) ABSTRACT

Various methods and systems are provided for radio frequency (RF) coils for magnetic resonance imaging (MRI). In one embodiment, an RF coil assembly for an MRI system includes a first end including a first set of flexible RF coil elements having a first shape, a second end including a second set of flexible RF coil elements having the first shape, and a central section extending between the first end and the second end and including a saddle shaped RF coil element. The first and second ends are bendable to the central section and the saddle shaped RF coil element is a different shape than the first shape. The saddle shaped RF coil element and each RF coil element of the first and second sets of RF coil elements includes a coupling electronics portion and at least two parallel, distributed capacitance wire conductors encapsulated and separated by a dielectric material.

### FIELD

Embodiments of the subject matter disclosed herein relate to medical diagnostic imaging, and in more particular, to systems for magnetic resonance imaging.

### BACKGROUND

Magnetic resonance imaging (MRI) is a medical imaging modality that can create images of the inside of a human body without using x-rays or other ionizing radiation. MRI systems include a superconducting magnet to create a strong, uniform, static magnetic field B₀. When an imaging subject is placed in the magnetic field B₀, the nuclear spins associated with the hydrogen nuclei in the imaging subject become polarized such that the magnetic moments associated with these spins become preferentially aligned along the direction of the magnetic field B₀, resulting in a small net magnetization along that axis. The hydrogen nuclei are excited by a radio frequency signal at or near the resonance frequency of the hydrogen nuclei, which add energy to the nuclear spin system. As the nuclear spins relax back to their rest energy state, they release the absorbed energy in the form of a radio frequency (RF) signal. This RF signal (or MR signal) is detected by one or more RF coil assemblies and is transformed into the image using reconstruction algorithms.

In order to detect the RF signals emitted by the body of the patient, an RF coil assembly is often positioned proximate anatomical features to be imaged by the MRI system. An image quality of images produced by the MRI system is influenced by an ability of the RF coil assembly to closely conform to the contours of the body of the patient.

### BRIEF DESCRIPTION

In one embodiment, an RF coil assembly for an MRI system includes a first end including a first set of flexible RF coil elements having a first shape, a second end including a second set of flexible RF coil elements having the first shape, and a central section extending between the first end and the second end and including a saddle shaped RF coil element. The first and second ends are bendable to the central section and the saddle shaped RF coil element is a different shape than the first shape. The saddle shaped RF coil element and each RF coil element of the first and second sets of RF coil elements includes a coupling electronics portion and at least two parallel, distributed capacitance wire conductors encapsulated and separated by a dielectric material.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 is a block diagram of an MRI system according to an exemplary embodiment.

FIG. 2 shows a view of an outer side of an RF coil assembly for an MRI system according to an exemplary embodiment.

FIGS. 3-8 show front views of a patient wearing the RF coil assembly of FIG. 2 in different configurations.

FIG. 9 shows an outer side of an RF coil assembly for an MRI system according to an exemplary embodiment.

FIG. 10 shows an outer side of an RF coil assembly for an MRI system according to an exemplary embodiment.

FIGS. 11A and 11B schematically show RF coils of an RF coil array coupled to a controller unit according to exemplary embodiments.

### DETAILED DESCRIPTION

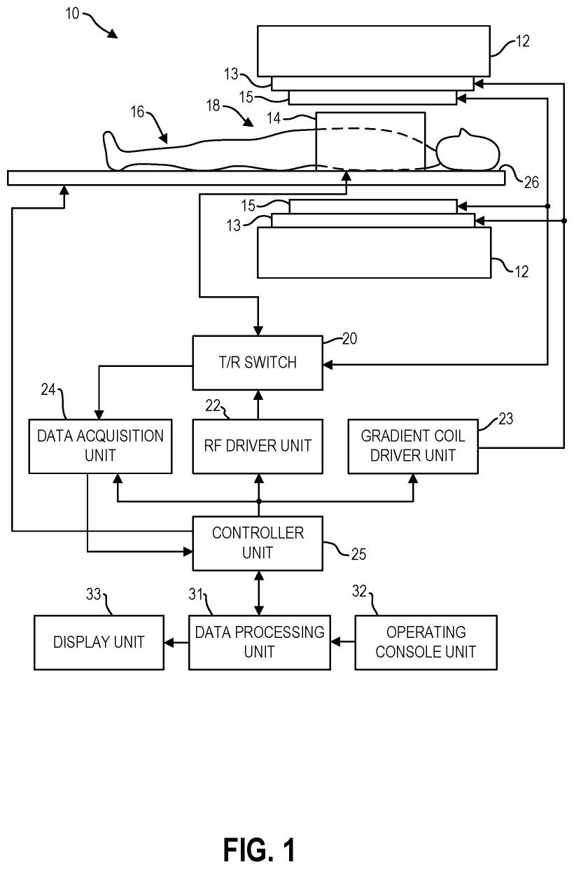

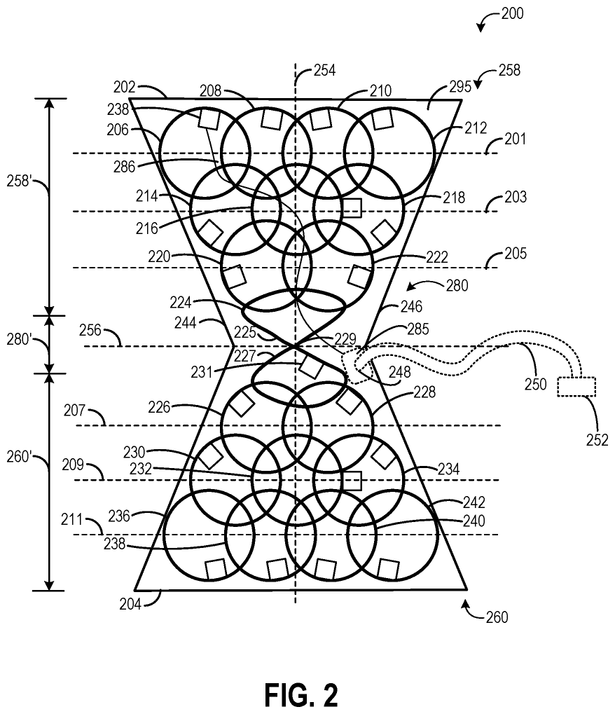

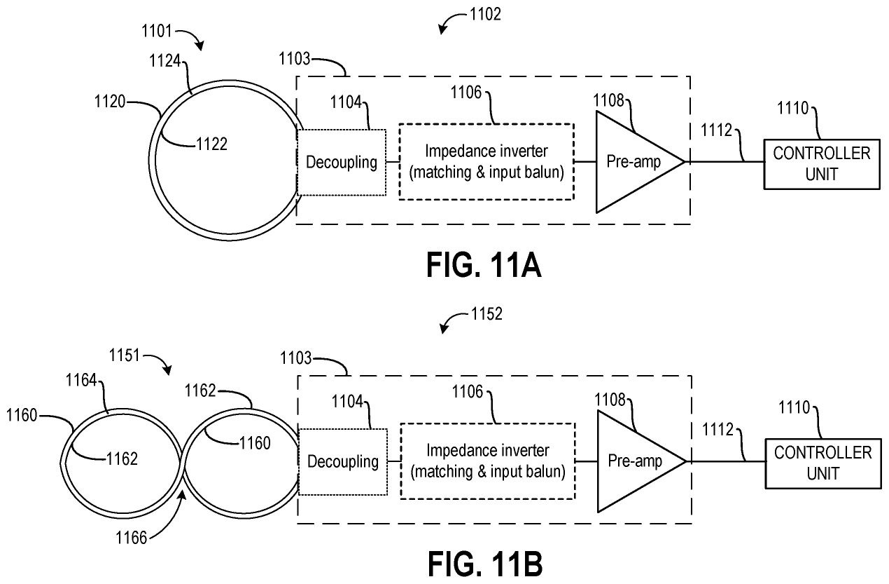

The following description relates to various embodiments of a radio frequency (RF) coil assembly for magnetic resonance imaging (MRI). An MRI system, such as the MRI system shown by FIG. 1, includes a flexible RF coil assembly, such as the RF coil assemblies shown by FIGS. 2, 9 and 10. The RF coils in the RF coil assembly are configured with coupling electronics and distributed capacitance wire conductors, as described with reference to FIGS. 11A and 11B, such that each RF coil is transparent to each other RF coil. In this way, the RF coils may be positioned with varying amounts of overlap, bent or curved relative to each other, etc., without compromising RF coil sensitivity and image quality. As such, the RF coils of the RF coil assembly may be positioned on flexible material, such as fabric, so that the ends of the RF coil assembly may be positioned against the body of the patient and wrapped around the patient in order to image portions of the body that are difficult to image with rigid RF coil assemblies, such as the shoulder. Because the RF coils include the coupling electronics and distributed capacitance wire conductors, sections of the RF coil assembly may move and/or overlap relative to each other without degradation of MR signals transmitted to the MRI system by the RF coils.

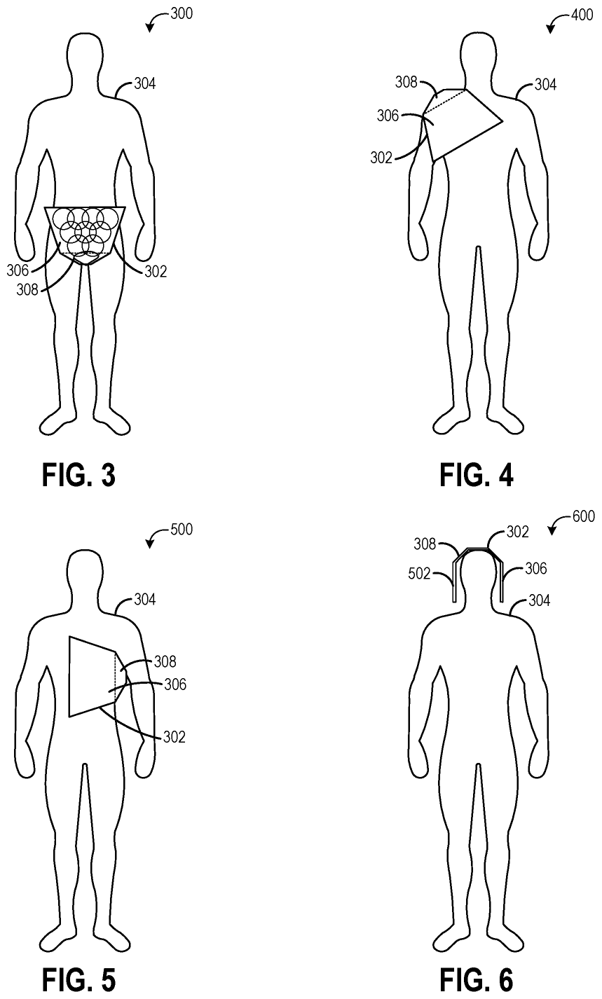

The RF coils described herein may be shaped as circular loops of distributed capacitance wire, which may facilitate desired coil sensitivity, maximize signal to noise ratio at depth, allow for parallel imaging, and provide other benefits. However, when the circular RF coils described herein are folded or bent to a relatively large extent at a central axis of the RF coils, such that RF coils are normal to the B₀ field, the sensitivity of the RF coils may decrease, which may reduce image quality. Thus, according to embodiments disclosed herein, rather than using circular, loop shaped RF coils at areas of the RF coil assembly that are likely to be subject to bending or folding during imaging, saddle shaped RF coils may be positioned in the areas of the RF coil assembly that are likely to be subject to bending or folding during imaging. For example, the RF coil assembly may be shaped as a bowtie, with two symmetrical flaps that are joined by a narrowed central section. The narrowed central section may serve as a bending region, where the bowtie RF coil assembly is configured to be bent or folded in order to closely conform to contours of the patient being imaged, such as the top of a shoulder, as shown by the various imaging configurations of FIGS. 3-8. The RF coil(s) at the central section may be saddle shaped RF coils. A saddle shaped RF coil may be a twisted loop that is formed by twisting a larger circular coil on itself one time in order to form a figure-eight. The saddle shaped RF coil may be positioned with its twist (also referred to as an intersection region of the RF coil) at a central axis of the RF coil assembly where bending of the RF coil assembly is expected to occur. Due to the saddle/figure-eight shape, the saddle RF coil may not decrease in sensitivity if bent at or near the center of the saddle such that the loops of the saddle are collinear with the B₀ field.

FIG. 1 illustrates a magnetic resonance imaging (MRI) apparatus 10 that includes a magnetostatic field magnet unit 12, a gradient coil unit 13, an RF coil unit 14, an RF body or volume coil unit 15, a transmit/receive (T/R) switch 20, an RF driver unit 22, a gradient coil driver unit 23, a data acquisition unit 24, a controller unit 25, a patient table or bed 26, a data processing unit 31, an operating console unit 32, and a display unit 33. In some embodiments, the RF coil unit 14 is a surface coil, which is a local coil typically placed proximate to the anatomy of interest of a subject 16. Herein, the RF body coil unit 15 is a transmit coil that transmits RF signals, and the local surface RF coil unit 14 receives the MR signals. As such, the transmit body coil (e.g., RF body coil unit 15) and the surface receive coil (e.g., RF coil unit 14) are separate but electromagnetically coupled components. The MRI apparatus 10 transmits electromagnetic pulse signals to the subject 16 placed in an imaging space 18 with a static magnetic field formed to perform a scan for obtaining magnetic resonance signals from the subject 16. One or more images of the subject 16 can be reconstructed based on the magnetic resonance signals thus obtained by the scan.

The magnetostatic field magnet unit 12 includes, for example, an annular superconducting magnet, which is mounted within a toroidal vacuum vessel. The magnet defines a cylindrical space surrounding the subject 16 and generates a constant primary magnetostatic field B₀.

The MRI apparatus 10 also includes a gradient coil unit 13 that forms a gradient magnetic field in the imaging space 18 so as to provide the magnetic resonance signals received by the RF coil arrays with three-dimensional positional information. The gradient coil unit 13 includes three gradient coil systems, each of which generates a gradient magnetic field along one of three spatial axes perpendicular to each other, and generates a gradient field in each of a frequency encoding direction, a phase encoding direction, and a slice selection direction in accordance with the imaging condition. More specifically, the gradient coil unit 13 applies a gradient field in the slice selection direction (or scan direction) of the subject 16, to select the slice; and the RF body coil unit 15 or the local RF coil arrays may transmit an RF pulse to a selected slice of the subject 16. The gradient coil unit 13 also applies a gradient field in the phase encoding direction of the subject 16 to phase encode the magnetic resonance signals from the slice excited by the RF pulse. The gradient coil unit 13 then applies a gradient field in the frequency encoding direction of the subject 16 to frequency encode the magnetic resonance signals from the slice excited by the RF pulse.

The RF coil unit 14 is disposed, for example, to enclose the region to be imaged of the subject 16. In some examples, the RF coil unit 14 may be referred to as the surface coil or the receive coil. In the static magnetic field space or imaging space 18 where a static magnetic field B₀ is formed by the magnetostatic field magnet unit 12, the RF coil unit 15 transmits, based on a control signal from the controller unit 25, an RF pulse that is an electromagnet wave to the subject 16 and thereby generates a high-frequency magnetic field B₁. This excites a spin of protons in the slice to be imaged of the subject 16. The RF coil unit 14 receives, as a magnetic resonance signal, the electromagnetic wave generated when the proton spin thus excited in the slice to be imaged of the subject 16 returns into alignment with the initial magnetization vector. In some embodiments, the RF coil unit 14 may transmit the RF pulse and receive the MR signal. In other embodiments, the RF coil unit 14 may only be used for receiving the MR signals, but not transmitting the RF pulse.

The RF body coil unit 15 is disposed, for example, to enclose the imaging space 18, and produces RF magnetic field pulses orthogonal to the main magnetic field B₀ produced by the magnetostatic field magnet unit 12 within the imaging space 18 to excite the nuclei. In contrast to the RF coil unit 14, which may be disconnected from the MRI apparatus 10 and replaced with another RF coil unit, the RF body coil unit 15 is fixedly attached and connected to the MRI apparatus 10. Furthermore, whereas local coils such as the RF coil unit 14 can transmit to or receive signals from only a localized region of the subject 16, the RF body coil unit 15 generally has a larger coverage area. The RF body coil unit 15 may be used to transmit or receive signals to the whole body of the subject 16, for example. Using receive-only local coils and transmit body coils provides a uniform RF excitation and good image uniformity at the expense of high RF power deposited in the subject. For a transmit-receive local coil, the local coil provides the RF excitation to the region of interest and receives the MR signal, thereby decreasing the RF power deposited in the subject. It should be appreciated that the particular use of the RF coil unit 14 and/or the RF body coil unit 15 depends on the imaging application.

The T/R switch 20 can selectively electrically connect the RF body coil unit 15 to the data acquisition unit 24 when operating in receive mode, and to the RF driver unit 22 when operating in transmit mode. Similarly, the T/R switch 20 can selectively electrically connect the RF coil unit 14 to the data acquisition unit 24 when the RF coil unit 14 operates in receive mode, and to the RF driver unit 22 when operating in transmit mode. When the RF coil unit 14 and the RF body coil unit 15 are both used in a single scan, for example if the RF coil unit 14 is configured to receive MR signals and the RF body coil unit 15 is configured to transmit RF signals, then the T/R switch 20 may direct control signals from the RF driver unit 22 to the RF body coil unit 15 while directing received MR signals from the RF coil unit 14 to the data acquisition unit 24. The coils of the RF body coil unit 15 may be configured to operate in a transmit-only mode or a transmit-receive mode. The coils of the local RF coil unit 14 may be configured to operate in a transmit-receive mode or a receive-only mode.

The RF driver unit 22 includes a gate modulator (not shown), an RF power amplifier (not shown), and an RF oscillator (not shown) that are used to drive the RF coils (e.g., RF coil unit 15) and form a high-frequency magnetic field in the imaging space 18. The RF driver unit 22 modulates, based on a control signal from the controller unit 25 and using the gate modulator, the RF signal received from the RF oscillator into a signal of predetermined timing having a predetermined envelope. The RF signal modulated by the gate modulator is amplified by the RF power amplifier and then output to the RF coil unit 15.

The gradient coil driver unit 23 drives the gradient coil unit 13 based on a control signal from the controller unit 25 and thereby generates a gradient magnetic field in the imaging space 18. The gradient coil driver unit 23 includes three systems of driver circuits (not shown) corresponding to the three gradient coil systems included in the gradient coil unit 13.

The data acquisition unit 24 includes a pre-amplifier (not shown), a phase detector (not shown), and an analog/digital converter (not shown) used to acquire the magnetic resonance signals received by the RF coil unit 14. In the data acquisition unit 24, the phase detector phase detects, using the output from the RF oscillator of the RF driver unit 22 as a reference signal, the magnetic resonance signals received from the RF coil unit 14 and amplified by the pre-amplifier, and outputs the phase-detected analog magnetic resonance signals to the analog/digital converter for conversion into digital signals. The digital signals thus obtained are output to the data processing unit 31.

The MRI apparatus 10 includes a table 26 for placing the subject 16 thereon. The subject 16 may be moved inside and outside the imaging space 18 by moving the table 26 based on control signals from the controller unit 25.

The controller unit 25 includes a computer and a recording medium on which a program to be executed by the computer is recorded. The program when executed by the computer causes various parts of the apparatus to carry out operations corresponding to pre-determined scanning. The recording medium may comprise, for example, a ROM, flexible disk, hard disk, optical disk, magneto-optical disk, CD-ROM, or non-volatile memory card. The controller unit 25 is connected to the operating console unit 32 and processes the operation signals input to the operating console unit 32 and furthermore controls the table 26, RF driver unit 22, gradient coil driver unit 23, and data acquisition unit 24 by outputting control signals to them. The controller unit 25 also controls, to obtain a desired image, the data processing unit 31 and the display unit 33 based on operation signals received from the operating console unit 32.

The operating console unit 32 includes user input devices such as a touchscreen, keyboard and a mouse. The operating console unit 32 is used by an operator, for example, to input such data as an imaging protocol and to set a region where an imaging sequence is to be executed. The data about the imaging protocol and the imaging sequence execution region are output to the controller unit 25.

The data processing unit 31 includes a computer and a recording medium on which a program to be executed by the computer to perform predetermined data processing is recorded. The data processing unit 31 is connected to the controller unit 25 and performs data processing based on control signals received from the controller unit 25. The data processing unit 31 is also connected to the data acquisition unit 24 and generates spectrum data by applying various image processing operations to the magnetic resonance signals output from the data acquisition unit 24.

The display unit 33 includes a display device and displays an image on the display screen of the display device based on control signals received from the controller unit 25. The display unit 33 displays, for example, an image regarding an input item about which the operator inputs operation data from the operating console unit 32. The display unit 33 also displays a two-dimensional (2D) slice image or three-dimensional (3D) image of the subject 16 generated by the data processing unit 31.

Conventional RF coils may include acid etched copper traces (loops) on printed circuit boards (PCBs) with lumped electronic components (e.g., capacitors, inductors, baluns, resistors, etc.), matching circuitry, decoupling circuitry, and pre-amplifiers. Such a configuration is typically very bulky, heavy, and rigid, and requires relatively strict placement of the coils relative to each other in an array (e.g., a set) to prevent coupling interactions among coil elements that may degrade image quality. As such, conventional RF coils and RF coil arrays lack flexibility and hence may not conform to patient anatomy, degrading imaging quality and patient comfort.

Thus, according to embodiments disclosed herein, an RF coil assembly, such as RF coil unit 14, may include distributed capacitance wire conductors formed as loops or other shapes rather than copper traces on PCBs with lumped electronic components. As a result, the RF coil assembly may be lightweight and flexible, allowing placement in low cost, lightweight, waterproof, and/or flame retardant fabrics or materials. The coupling electronics portion coupling the loop portion of the RF coil (e.g., the distributed capacitance wire) may be miniaturized and utilize a low input impedance pre-amplifier, which is optimized for high source impedance (e.g., due to impedance matching circuitry) and allows flexible overlaps among coil elements in an RF coil array (e.g., RF coil set). Further, the RF coil-interfacing cable between the RF coils and system processing components may be flexible and include integrated transparency functionality in the form of distributed baluns, which allows rigid electronic components to be avoided and aids in spreading of the heat load.

The RF coil assemblies described herein may be structured for imaging specific anatomical features of a patient that are often difficult to image with rigid (e.g., inflexible) RF coil arrays. Specifically, the RF coil assemblies of the present disclosure include a first end, a second end, and a central section joining the first end to the second end. The first end, second end, and central section are each formed of a flexible material and may each include at least one RF coil. The RF coils of the first end, second end, and central section are electrically coupled to a common output (e.g., a single coil-interfacing cable or cable harness) that is electrically coupleable to the MRI apparatus 10. Each of the first end, second end, and central section may be wrapped around the anatomical feature of interest to be imaged by the MRI apparatus 10. Specifically, the RF coil assembly may be coupled to the patient proximate to the groin, shoulder, head, neck, or other region of the patient, with the first end typically positioned at a first (e.g., front) side of the patient, the second end positioned at a second (e.g., rear) side of the patient, and the central section positioned at an intervening anatomical region, such as the perineum of the patient, the top of the shoulder, the side of an arm, etc. In this way, the RF coil assembly may be utilized to image anatomy of the patient that is curved, spans multiple (and often perpendicular) planes, or is otherwise difficult to image with traditional RF coils.

Imaging anatomy disposed in areas having a high degree of curvature (e.g., shoulder, head, and groin) is often difficult and/or uncomfortable for the patient with conventional, rigid RF coil arrays due to the varying size and/or curvature of the anatomy from patient to patient. Conventional, rigid RF coil arrays may be unable to closely conform to the anatomy of the patient. However, the flexible RF coil assembly disclosed herein may be fitted to a wide variety of patients of different sizes (e.g., weights, heights, etc.). Further, the RF coil assembly disclosed herein may increase a signal-to-noise ratio (SNR) of the images produced by the MRI apparatus 10 relative to conventional RF coils due to the ability of the sections of the RF coil assembly to wrap around the anatomy of the patient, enabling the RF coils to be positioned closer to the body of the patient. The ability of the RF coil assembly to fit to a wider variety of patients may decrease an imaging cost of the MRI apparatus 10 (e.g., by reducing a number of different RF coil assemblies utilized to image patients via the MRI apparatus 10) and may increase the imaging quality of the MRI apparatus 10 (e.g., by increasing the SNR).

Turning now to FIG. 2, an RF coil assembly 200 according to a first exemplary embodiment is shown. RF coil assembly 200 (which may be referred to herein as a wearable RF coil assembly) may be similar to the RF coil unit 14 described above with reference to FIG. 1. For example, RF coil assembly 200 may be electrically coupleable to an MRI apparatus (e.g., MRI apparatus 10 of FIG. 1 and described above) for imaging one or more anatomical features of a patient. As will be explained in more detail below with respect to FIGS. 3-8, RF coil assembly 200 may be utilized in order to image various anatomical features of a patient, including but not limited to the prostate, groin, shoulder, neck, chest, head, leg, and ankle.

The RF coil assembly 200 is a flexible RF coil assembly that may deform (e.g., bend, twist, etc.) in multiple different directions. The RF coil assembly 200 is shaped as a bowtie and thus may be referred to as a bowtie RF coil assembly. The RF coil assembly 200 includes a first end 258 and a second end 260, with the first end 258 configured to couple to (e.g., wrap around) a first side of the patient, and with the second end 260 configured to couple to (e.g., wrap around) a second side of the patient, at least during some imaging protocols. As will be explained in more detail below, RF coil assembly 200 may be positioned to image a pelvic region, a shoulder, a chest, a head, or other anatomy, and thus the first side and second side of the patient may depend on how the RF coil assembly 200 is positioned. For example, when RF coil assembly 200 is positioned to image a pelvic region (as shown in FIG. 3), the first side of the patient may be an anterior side and the second side of the patient may be a posterior side.

As mentioned above, the RF coil assembly 200 is shaped as a bowtie, with two symmetric flaps (akin to the loops of the bowtie) joined by a narrowed central section (akin to the knot of the bowtie). The first end 258 may define the first flap and the second end 260 may define the second flap. A central section 280 of the RF coil assembly 200, described further below, extends between first end 258 and second end 260 of the RF coil assembly 200 and defines the narrowed central section of the bowtie. First end 258, second end 260, and central section 280 may be defined relative to a length of RF coil assembly 200. FIG. 2 includes arrows to the left of RF coil assembly 200 to aid in visualization of the extent of each of first end 258, second end 260, and central section 280. As shown, first end 258 extends along first end length 258′, second end 260 extends along second end length 260′, and central section 280 extends along central section length 280′. However, it is to be appreciated that the exact regions where first end 258 terminates and central section 280 begins (and where central section 280 terminates and second end 260 begins) are exemplary and that first end 258, second end 260, and central section 280 may have different lengths without departing from the scope of this disclosure.

The first end 258, second end 260, and central section 280 are each moveable (e.g., pivotable and/or bendable) relative to each other. For example, first end 258 and second end 260 may bend relative to the central section 280 to a position in which the first end 258 and second end 260 are approximately perpendicular to the central section 280. By configuring the RF coil assembly 200 to be flexible in this way, the first end 258 and second end 260 are bendable to the central section 280. However, in the view shown by FIG. 2, the RF coil assembly 200 is in a flattened configuration in which the RF coil assembly 200 is not coupled to the patient. In the flattened configuration, each of the first end 258, second end 260, and central section 280 are relatively flat (e.g., not moved, bent, etc. relative to each other) and planar (e.g., positioned parallel with each other along a same plane). The first end 258, second end 260, and central section 280 may be referred to herein collectively as a body of the RF coil assembly 200, and may be worn by the patient for imaging of the patient via the MRI system.

The flattened configuration of the RF coil assembly 200 shown by FIG. 2 shows an outer side of the RF coil assembly 200 comprising an outer surface 295. The outer side of the RF coil assembly 200 is a side that is not in direct contact with the body of the patient during conditions in which the RF coil assembly 200 is coupled to (e.g., worn by) the patient. Further, the outer surfaces, such as outer surface 295, are not in direct contact with the body of the patient during conditions in which the RF coil assembly 200 is coupled to the patient. The RF coil assembly 200 further includes an inner side (not visible in FIG. 2) configured to be in direct contact (e.g., face-sharing contact, with no other components positioned between) with the body of the patient during conditions in which the RF coil assembly 200 is coupled to the patient (e.g., for imaging via the MRI system). In some examples, the inner side may include one or more inner surfaces comprising pads, cushions, etc., positioned to increase patient comfort during conditions in which the RF coil assembly 200 is coupled to the patient. In this way, the RF coils (described in more detail below) of the RF coil assembly 200 may be coupled to the outer surface and the inner surface may be positioned on an opposite side of the outer surface relative to the RF coils. While not shown in FIG. 2 for clarity, in some examples, a cover layer may be presented over the outer-facing sides of the RF coils to protect the RF coils from dirt, debris, etc.

First end 258 of RF coil assembly 200 includes nine RF coils (e.g., first RF coil 206, second RF coil 208, third RF coil 210, fourth RF coil 212, fifth RF coil 214, sixth RF coil 216, seventh RF coil 218, eighth RF coil 220, and ninth RF coil 222), the central section 280 includes one RF coil (e.g., tenth RF coil 224), and the second end 260 includes nine RF coils (e.g., eleventh RF coil 226, twelfth RF coil 228, thirteenth RF coil 230, fourteenth RF coil 232, fifteenth RF coil 234, sixteenth RF coil 236, seventeenth RF coil 238, eighteenth RF coil 240, and nineteenth RF coil 242). In total, the RF coil assembly 200 includes nineteen RF coils. The RF coils described herein may also be referred to as RF coil elements. The nine RF coils of the first end 258 are arranged into three separate rows and may be referred to herein collectively as an RF coil set, with a first row positioned furthest from the central section 280 including four coils centered along axis 201, a second row adjacent to the first row including three coils centered along axis 203, and a third row positioned closest to the central section 280 including two coils centered along axis 205. Specifically, first RF coil 206, second RF coil 208, third RF coil 210, and fourth RF coil 212 of the first end 258 are each positioned along axis 201 and are bisected by the axis 201, fifth RF coil 214, sixth RF coil 216, and seventh RF coil 218 are each positioned along axis 203 and are bisected by the axis 203, and eighth RF coil 220 and ninth RF coil 222 are each positioned along axis 205 and are bisected by the axis 205.

The RF coils of the second row of the first end 258 may overlap the RF coils of the first row of the first end 258 and the third row of the first end 258. The RF coils of the second row are positioned between the RF coils of the first row and the RF coils of the third row of the first end 258. Specifically, as shown by FIG. 2, fifth RF coil 214 of the second row of the first end 258 overlaps the first RF coil 206 of the first row and the eighth RF coil 220 of the third row of the first end 258, sixth RF coil 216 overlaps the second RF coil 208 and third RF coil 210 of the first row of the first end 258 in addition to the eighth RF coil 220 and ninth RF coil 222 of the third row of the first end 258, and seventh RF coil 218 overlaps the third RF coil 210 and fourth RF coil 212 of the first row of the first end 258 in addition to the ninth RF coil 222 of the third row of the first end 258. As described herein, overlapping RF coils refers to a loop portion of an RF coil encircling and/or directly contacting at least some of a loop portion of another RF coil. For example, as shown by FIG. 2, first RF coil 206 overlaps with second RF coil 208 and fifth RF coil 214. However, first RF coil 206 does not overlap with third RF coil 210, fourth RF coil 212, sixth RF coil 216, seventh RF coil 218, eighth RF coil 220, ninth RF coil 222, or any of the RF coils of the central section 280 or second end 260. Further, none of the RF coils of the first end 258 overlap with any of the RF coils of the second end 260 (e.g., the first end 258 is spaced apart from the second end 260 by the central section 280 such that the RF coils of the first end 258 do not overlap the RF coils of the second end 260).

The nine RF coils of the second end 260 are arranged into three separate overlapping rows similar to the nine RF coils of the first end 258 and may also be referred to herein collectively as an RF coil set, with a first row positioned further from the central section 280 including four coils centered along axis 211, a second row positioned closer to the central section 280 including three coils centered along axis 209, and a third row positioned closest to the central section 280 and including two coils centered along axis 207. The RF coils of the second end 260 are arranged in a symmetric manner to the RF coils of the first end 258, and thus the description of the arrangement of the RF coil elements in the overlapping rows of first end 258 applies to the arrangement of the RF coil elements in the overlapping rows of the second end 260.

The central section 280 includes only one RF coil, tenth RF coil 224. Tenth RF coil 224 is a saddle coil, in contrast to the RF coils of the first end 258 and second end 260, which are circular loop coils. A saddle coil may be a twisted loop that includes a loop coil that has been twisted to form a figure-eight shape, with two loops that meet at an intersecting region in a center of the coil. As shown, tenth RF coil 224 includes a first loop 225 and a second loop 227 that meet at an intersecting region 229. The first loop 225 and second loop 227 are comprised of a continuous set of parallel wires, and, at the intersecting region 229, a segment of the wire set is positioned on top of another segment of the wire set. The segments of wire sets do not touch at the intersecting region, due to the wires being encapsulated in an insulating material, as will be described in more detail below.

Tenth RF coil 224 extends into both first end 258 and second end 260 to overlap with RF coils of both the first end 258 and the second end 260. For example, tenth RF coil 224 overlaps eighth RF coil 220 and ninth RF coil 222 of the first end 258 (e.g., first loop 225 overlaps eighth RF coil 220 and ninth RF coil 222) and also overlaps eleventh RF coil 226 and twelfth RF coil 228 of second end 260 (e.g., second loop 227 overlaps eleventh RF coil 226 and twelfth RF coil 228). Tenth RF coil 224 may be sized and/or shaped in order to provide a desired amount of overlap with the RF coils of the first end and second end as described above. In some embodiments, first loop 225 and second loop 227 may be the same size and shape. In other embodiments, first loop 225 and second loop 227 may be of different size or shape

Tenth RF coil 224 may be centered along a central transverse axis 256 of the RF coil assembly 200. As shown in FIG. 2, intersecting region 229 of tenth RF coil 224 is positioned along central transverse axis 256. Intersecting region 229 is also positioned along a central longitudinal axis 254 of the RF coil assembly 200. Central transverse axis 256 is perpendicular to central longitudinal axis 254. Central transverse axis 256 may define a first axis of symmetry of the RF coil assembly 200 and central longitudinal axis 254 may define a second axis of symmetry, at least with respect to the shape of the outer surface 295 and positioning of the loops of the RF coil elements. Central transverse axis 256 is centered between a distal edge 202 of the first end 258 and a distal edge 204 of the second end 260. Central transverse axis 254 bisects each of a first side edge 244 and a second side edge 246 of the RF coil assembly during conditions in which the RF coil assembly 200 is in the flattened configuration (e.g., as shown by FIG. 2). While RF coil assembly 200 as shown in FIG. 2 includes two axes of symmetry, in some examples RF coil assembly 200 may have fewer axes of symmetry. For example, first end 258 and second end 260 may not be symmetric relative to central transverse axis 256. Instead, first end 258 may be larger or smaller than second end 260, first end 258 may include more or fewer RF coils than second end 260, and so forth.

In the example shown by FIG. 2, the RF coils of the RF coil assembly 200 at the first end 258 have a same diameter and same eccentricity as the RF coils of the RF coil assembly 200 at the second end 260. For example, all the RF coils in the RF coil assembly 200 other than the tenth RF coil 225 may have the same diameter and same eccentricity. In one example, the eccentricity of the RF coils of the first end 258 and second end 260 is 0 (e.g., the RF coils at the first end 258 and second end 260 have a circular shape). In other examples, the eccentricity of the RF coils of the first end 258 and second end 260 may be a different value (e.g., 0.5, 0.6, etc.). In some embodiments, a diameter of RF coils of the first end 258 and second end 260 may be 11 centimeters, or other suitable diameter depending on the size of the patient that is to be imaged (e.g., larger patients may be imaged with an RF coil assembly having larger RF coil elements while smaller patients may be imaged with an RF coil assembly having smaller RF coil elements). In some examples, the saddle RF coil (e.g., tenth RF coil 224) may have an area that is two-thirds to twice the area of an area of the RF coils of the first and second ends, which may provide a similar sensitivity as the RF coils of the first and second ends at the same depth.

In some examples, one or more of the RF coils of the RF coil assembly 200 may have a different diameter than other RF coils of the RF coil assembly 200. For example, the loops of the RF coil of the central section 280 (tenth RF coil 224) may have a different diameter (e.g., a smaller diameter) than the diameter of the RF coils of the first end 258 and/or second end 260. In another example, RF coils of the first end 258 may have a different diameter than RF coils of the second end 260. In yet another example, one or more of the RF coils of the first end 258 may have a different diameter relative to other RF coils of the first end 258, and/or one or more RF coils of the second end 260 may have a different diameter relative to other RF coils of the second end 260.

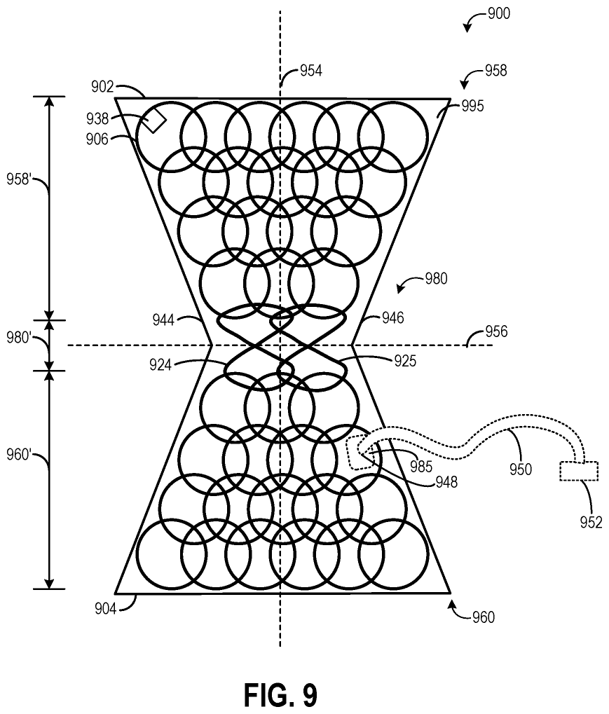

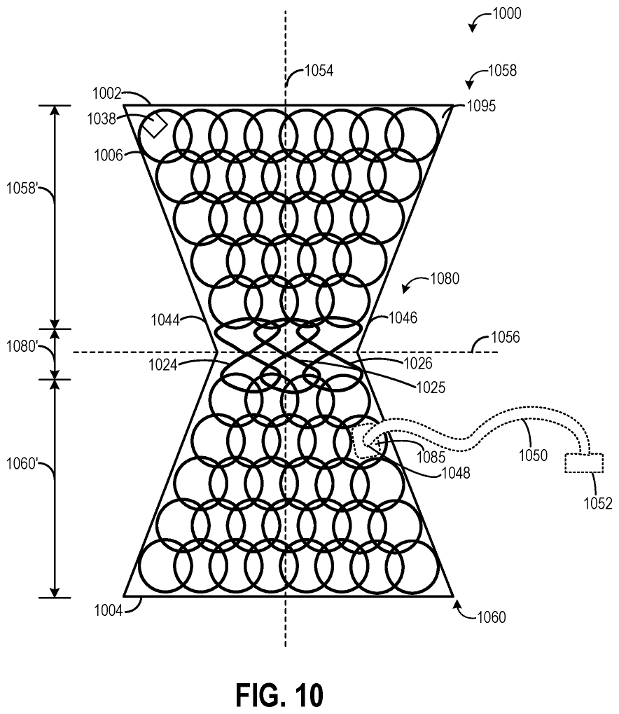

In some examples, the RF coil assembly 200 may include a different number of RF coils relative to the examples described above. For example, the first end 258 may include a different number of RF coils than nine RF coils (e.g., seven RF coils, eight RF coils, ten RF coils, etc.), the second end 260 may include a different number of RF coils than nine RF coils (e.g., seven RF coils, eight RF coils, ten RF coils, etc.), and/or the central section 280 may include a different number of RF coils than one RF coil (e.g., two RF coils, three RF coils, etc.). Additional details about higher density coil arrays are provided below with respect to FIGS. 9 and 10.

In some examples, the RF coil assembly 200 may include RF coils in a different arrangement relative to the example shown by FIG. 2. As one example, the RF coils of the first end 258, second end 260, and/or central section 280 may not be arranged in rows. For example, the first RF coil 206, second RF coil 208, third RF coil 210, and fourth RF coil 212 may not be arranged along axis 201. Instead, one or more of the first RF coil 206, second RF coil 208, third RF coil 210, and fourth RF coil 212 may be offset from the axis 201 by a different amount relative to at least one other RF coil of the first RF coil 206, second RF coil 208, third RF coil 210, and fourth RF coil 212. For example, first RF coil 206 and fourth RF coil 212 may be centered along axis 201, and second RF coil 208 and third RF coil 210 may be offset from the axis 201 (e.g., shifted toward or away from central section 280). Similarly, the fifth RF coil 214, sixth RF coil 216, and seventh RF coil 218 may not be aligned (e.g., centered) along axis 203, the eighth RF coil 220 and ninth RF coil 222 may not be aligned along axis 205, the tenth RF coil 224 may not be aligned along central transverse axis 256, etc.

As appreciated by FIG. 2, the first side edge 244 and second side edge 246 each slope inward toward central longitudinal axis 254 from distal edge 202 until central transverse axis 256. First side edge 244 and second side edge 246 also slope inward toward central longitudinal axis 254 from distal edge 204 until central transverse axis 256. In this way, RF coil assembly 200 includes a most-narrow region at central transverse axis 256, with the RF coil assembly 200 increasing gradually in width from the most-narrow region to each of the distal ends, creating a mirrored pyramid shape. By doing so, the central section 280 may more easily conform to patient anatomy when RF coil assembly 200 is positioned over curving anatomy.

Although the RF coils of the RF coil assembly 200 are shown by FIG. 2, it should be noted that the RF coils may be embedded within a material of the RF coil assembly 200 and may not be visible to an observer (e.g., the patient or operator of the MRI system). The RF coils are shown by FIG. 2 in order to illustrate a relative positioning and arrangement of the RF coils with respect to the first end 258, second end 260, and central section 280. For example, each of the first end 258, second end 260, and central section 280 (e.g., the body of the RF coil assembly 200) may be formed of a flexible material that is transparent to RF signals, such as one or more layers of meta-aramid material (e.g., Nomex® fabric). The RF coils of the first end 258, second end 260, and/or central section 280 may be embedded within the flexible material in some examples (e.g., fully enclosed by one or more layers of the flexible material). In other examples, the RF coils may be fixedly coupled to the RF coil assembly. For example, the RF coils of the first end 258 may be stitched or otherwise fixed (e.g., mounted, glued, fastened, etc.) to the material of the first end 258, the RF coils of the second end 260 may be stitched or otherwise fixed to the material of the second end 260, and/or the RF coils of the central section 280 may be stitched or otherwise fixed to the material of the central section 280. Because the body of the RF coil assembly 200 (e.g., the first end 258, second end 260, and central section 280) is formed of the flexible material, the body may be configured to wrap around a hip or other anatomy of the subject to be imaged (e.g., the patient). For example, portions of each of the first end 258 and second end 260 may overlap across the hip of the patient during conditions in which the RF coil assembly 200 is coupled to the patient for imaging of the patient (e.g., as shown by FIGS. 3 and 8).

Further, each RF coil is coupled to corresponding coupling electronics (e.g., coupling electronics portions 238 coupled to first RF coil 206), and the corresponding coupling electronics (and the electrical wires coupled to the coupling electronics and/or RF coils) may be embedded within the flexible material along with the RF coils. For example, coupling electronics portion 238 of first RF coil 206 may be embedded within the material of first end 258. In other examples, the RF coils, coupling electronics, and/or electrical wires may be coupled (e.g., mounted) to the RF coil assembly 200 (e.g., mounted to first end 258, central section 280, and/or second end 260). The RF coils may bend and/or deform along with the flexible material without degradation of signals (e.g., RF signals) associated with the RF coils (e.g., signals used to image the patient with the MRI system via the RF coil assembly, as described above).

The RF coils of the first end 258, second end 260, and central section 280 are electrically coupled to a single output (e.g., a single coil-interfacing cable or cable harness) that is electrically coupleable to the MRI system. For example, FIG. 2 shows coil-interfacing cable 250 having an output connector 252 adapted to couple to the MRI system in order to transmit electrical signals from the RF coils of the RF coil assembly 200 to the MRI system. Each RF coil may be electrically coupled with the coil-interfacing cable 250 and output connector 252 via respective coupling electronics. Specifically, the coupling electronics of each RF coil (e.g., the RF coils of the first end 258, second end 260, and central section 280) may be electrically coupled to interface board 285 via wires, and interface board 285 may be electrically coupled with output connector 252 via coil-interfacing cable 250. For example, first RF coil 206 is electrically coupled to the interface board 285 via coupling electronics portion 238. Coupling electronics portion 238 may be electrically coupled to the interface board 285 via one or more wires (e.g., wire 286), and interface board 285 may transmit signals (e.g., electrical signals) from the coupling electronics portion 238 to the output connector 252 via coil-interfacing cable 250. In some examples, the wires may be embedded within the material of the RF coil assembly 200, and may extend toward the interface board 285 in order to electrically couple the coupling electronics of each RF coil with the interface board 285. Although the wire 286 extending from the coupling electronics portion 238 is shown in FIG. 2, the other wires (e.g., each RF coil of FIG. 2 includes a respective coupling electronics portion that is coupled to interface board via a respective wire) have been omitted for illustrative purposes.

Each RF coil, including tenth RF coil 224, may have only one coupling electronics portion. In particular, while tenth RF coil 224 is comprised of two loops, the two loops are formed from a single loop that is twisted to form the saddle/figure-eight shape. Because tenth RF coil 224 is comprised of one loop that is twisted into the saddle shape, tenth RF coil 224 only includes one coupling electronics portion, herein coupling electronics portion 231. In this way, coil sensitivity at the narrowed central section, which is configured to bend or fold when placed over certain anatomy during imaging, may be maintained via inclusion of the saddle coil. When the loops of the saddle coil are oriented close to normal to the B₀ field, their separate sensitivity is very low, so the output from the loops may be combined to generate a saddle-like element. The combination of the output from the loops may be combined in post-processing, if the loops were separate loops. However, knowing that the loops are expected to be in the collinear position relative to the B₀ field during imaging, the loops may be combined in hardware (e.g., forming the saddle coil). In doing so, fewer electronics and cabling is necessary for saddle RF coil relative to two separate circular/planar loop coils.

Coil-interfacing cable 250 may be electrically coupled to the interface board 285 via a port 248 (e.g., an opening). For example, coil-interfacing cable 250 may include a plurality of wires adapted to transmit electrical signals from the interface board 285 to the output connector 252. In one example, coil-interfacing cable 250 and interface board 285 may be integrated together as a single piece, with the interface board 285 embedded within the material of the RF coil assembly 200 and with the coil-interfacing cable 250 extending outward from the RF coil assembly 200. In other examples, port 248 may include a connector adapted to enable the coil-interfacing cable 250 to removably couple with the interface board 285. For example, coil-interfacing cable 250 may include an input connector shaped to couple with the connector at port 248. In this configuration, coil-interfacing cable 250 may be coupled to the interface board 285 (e.g., via the connector at the port 248) during conditions in which the RF coil assembly 200 is utilized to image the patient via the MRI system, and the coil-interfacing cable 250 may be de-coupled from the interface board 285 (e.g., removed from the RF coil assembly 200) for replacement, maintenance, etc.

The port 248 and/or interface board 285 may be positioned at a suitable location on RF coil assembly 200. Accordingly, port 248, interface board 285, coil interfacing cable 250, and output connector 252 are shown in dashed lines in FIG. 2 in order to signify that port 248 and interface board 285 (and hence cable 250 and connector 252) may be positioned elsewhere on RF coil assembly 200 without departing from the scope of the disclosure.

The coil-interfacing cable 250 extends in an outward direction from the port 248 and interface board 285 (e.g., a direction away from the outer surfaces of the outer side of RF coil assembly 200, such as outer surface 295), with each of the RF coils of the RF coil assembly 200 electrically coupled to the output connector 252 via the coil-interfacing cable 250 (e.g., via the coupling electronics and interface board 285 as described above). Port 248 may be open at the outer side of the RF coil assembly 200 (e.g., the side shown by FIG. 2) and may be closed at the inner side of the RF coil assembly 200. In some examples, the port 248 may be encircled by one or more RF coils.

In some examples, the RF coil assembly 200 may include more than one coil-interfacing cable. For example, RF coil assembly 200 may include two coil-interfacing cables similar to the coil-interfacing cable 250, with a first coil-interfacing cable electrically coupled to the RF coils of the second end 260, and with a second coil-interfacing cable electrically coupled to the RF coils of the first end 258. Further, one of the first coil-interfacing cable or second coil-interfacing cable may be electrically coupled to the RF coils of the central section 280. The first coil-interfacing cable and second coil-interfacing cable may each extend outward from the RF coil assembly 200 via separate ports of the RF coil assembly 200. As one example, the RF coil assembly 200 may include a first port and a second port similar each similar to port 248, with the first coil-interfacing cable extending outward from the first port and with the second coil-interfacing cable extending outward from the second port. The first port and second port may be offset from each other (e.g., spaced apart from each other by a length of the RF coil assembly 200). In one example, the first port and second port are each positioned at the central section 280. In another example, one or both of the first port and second port may be positioned at the second end 260 or first end 258 (e.g., the first port may be positioned at the first end 258 and the second port may be positioned at the second end 260). As another example, the first port may be positioned at the central section 280 and the second port may be positioned at the first end 260 or second end 258. Other examples are possible.

The first coil-interfacing cable and second coil-interfacing cable may each be electrically coupled to a same interface board in one example (e.g., interface board 285). In another example, the first coil-interfacing cable may be electrically coupled to a first interface board (e.g., similar to interface board 285), and the second coil-interfacing cable may be electrically coupled to a second interface board. The first interface board may be positioned at the first port and the second interface board may be positioned at the second port. In some examples, the first coil-interfacing cable and first interface board may be integrated together as a single piece, with the first interface board embedded within the material of the RF coil assembly 200 and with the first coil-interfacing cable electrically coupled to the first interface board and extending outward from the first port of the RF coil assembly 200. Similarly, the second coil-interfacing cable and second interface board may be integrated together as a single piece, with the second interface board embedded within the material of the RF coil assembly 200 and with the second coil-interfacing cable electrically coupled to the second interface board and extending outward from the second port of the RF coil assembly 200. In other examples, the first port may include a connector adapted to enable the first coil-interfacing cable to removably couple with the first interface board, and/or the second port may include a connector adapted to enable the second coil-interfacing cable to removably couple with the second interface board, similar to the example of coil-interfacing cable 250 and interface board 285 described above.

In yet another example, the RF coil assembly may include three coil-interfacing cables, with a first coil-interfacing cable electrically coupled to the RF coils of the second end 260, a second coil-interfacing cable electrically coupled to the RF coils of the first end 258, and a third coil-interfacing cable electrically coupled to the RF coils of the central section 280. The first coil-interfacing cable may extend outward from a first port of the RF coil assembly 200 (e.g., similar to port 248) and may be electrically coupled to a first interface board (e.g., interface board 285), the second coil-interfacing cable may extend outward from a second port of the RF coil assembly 200 and may be electrically coupled to a second interface board, and the third coil-interfacing cable may extend outward from a third port of the RF coil assembly 200 and may be electrically coupled to a third interface board. Similar to the example described above, two or more of the coil-interfacing cables may be electrically coupled to a same interface board in some examples, and/or one or more of the ports may be positioned at a different location of the RF coil assembly 200 (e.g., second end 260, first end 258, or central section 280) than one or more other ports of the RF coil assembly 200. Other examples are possible.

FIGS. 3-8 show one or more RF coil assemblies according to the present disclosure arranged on a patient. Referring first to FIG. 3, it shows a first configuration 300 of an RF coil assembly 302 on a patient 304. RF coil assembly 302 is a non-limiting example of RF coil assembly 200 and as such includes a first end 306, a second end (not visible in FIG. 3), and a central section 308. The second end of RF coil assembly 302 is not visible in FIG. 3, as the second end is positioned on an opposite side of patient 304 from first end 306. In the first configuration 300, RF coil assembly 302 is positioned at a groin/pelvic region of patient 304. Accordingly, first end 306 is positioned on/proximate a first side of the groin (e.g., an anterior side), the second end is positioned on/proximate a second side of the groin (e.g., a posterior side), and central section 308 wraps around a curved/intersecting region of the groin (e.g., the perineum).

The RF coils of first end 306 (and the second end) may remain substantially planar relative to other RF coils in first end 306 (or relative to RF coils of the second end), even as RF coil assembly 302 is wrapped around patient 304. In contrast, the RF coil elements of central section 308 (e.g., one or more saddle RF coils) are substantially non-planar when RF coil assembly 302 is wrapped around patient 304. As used herein, substantially may include being the same (e.g., in the same plane) or within a threshold amount, such as within 5% of a given reference point.

FIG. 4 shows a second configuration 200 of RF coil assembly 302 on patient 304. In the second configuration 400, RF coil assembly 302 is positioned at a shoulder region of patient 304. Accordingly, first end 306 is positioned on/proximate a first side of the shoulder (e.g., an anterior side), the second end (which is not visible in FIG. 4) is positioned on/proximate a second side of the shoulder (e.g., a posterior side), and central section 308 wraps around a curved/intersecting region of the shoulder (e.g., the top of the shoulder).

FIG. 5 shows a third configuration 500 of RF coil assembly 302 on patient 304. In the third configuration 500, RF coil assembly 302 is positioned at a chest of patient 304, and is specifically positioned to image the patient's heart. Accordingly, first end 306 is positioned on/proximate a first side of the chest (e.g., an anterior side), the second end (which is not visible in FIG. 5) is positioned on/proximate a second side of the chest (e.g., a posterior side), and central section 308 wraps around a curved/intersecting region of the chest (e.g., a side of the rib cage under the arm of the patient).

FIG. 6 shows a fourth configuration 600 of RF coil assembly 302 on patient 304. In the fourth configuration 600, RF coil assembly 302 is positioned on a head of patient 304. Accordingly, first end 306 is positioned on/proximate a first side of the head (e.g., a left side), the second end 502 is positioned on/proximate a second side of the head (e.g., a right side), and central section 308 wraps around a curved/intersecting region of the head (e.g., the top of the head).

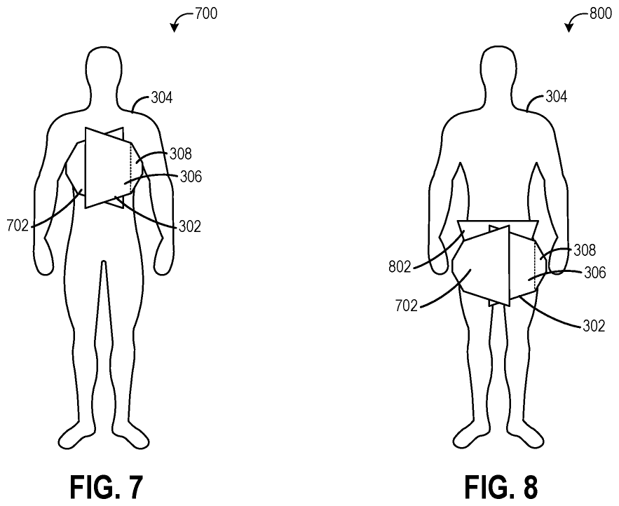

FIGS. 7 and 8 show example configurations where more than one RF coil assembly is used to image a patient. FIG. 7 shows a fifth configuration 700 where RF coil assembly 302 and a second RF coil assembly 702 are positioned on patient 304. In the fifth configuration 700, RF coil assembly 302 is positioned at a first half (e.g., a left half) of a chest of patient 304. Accordingly, first end 306 is positioned on/proximate a first side of the chest (e.g., an anterior side), the second end (which is not visible in FIG. 7) is positioned on/proximate a second side of the chest (e.g., a posterior side), and central section 308 wraps around a curved/intersecting region of the chest (e.g., a left side of the rib cage under the left arm of the patient). Second RF coil assembly 702 is positioned at a second half (e.g., a right half) of the chest of patient 304. Thus, a first end of second RF coil assembly 702 is positioned on the first side of the chest, a second end of second RF coil assembly 702 is positioned on the second side of the chest, and a central section of second RF coil assembly wraps around a curved/intersecting region of the chest (e.g., a right side of the rib cage under the right arm of the patient). RF coil assembly 302 and second RF coil assembly 702 may overlap each other on the first side of the chest (e.g., at the sternum) and on the second side of the chest (e.g., along the spine). Further, second RF coil assembly 702 may include a similar structure (e.g., similar number and/or arrangement of RF coils) relative to RF coil assembly 302.

FIG. 8 shows a sixth configuration 800 where RF coil assembly 302, second RF coil assembly 702, and a third RF coil assembly 802 are positioned on patient 304. In the sixth configuration 800, the RF coil assemblies are positioned at a groin/pelvic region and around the hips of patient 304. As shown, third RF coil assembly 802 includes a first end positioned on/proximate a first side of the groin (e.g., an anterior side), a second end positioned on/proximate a second side of the groin (e.g., a posterior side), and a central section that wraps around a curved/intersecting region of the groin (e.g., the perineum). RF coil assembly 302 is positioned on third RF coil assembly 802 at a first half (e.g., the left half) of the groin/pelvic region of patient 304. Accordingly, first end 306 is positioned on/proximate the first side of the groin, the second end is positioned on/proximate the second side of the groin, and central section 308 wraps around a curved/intersecting region of the groin (e.g., the left hip). Second RF coil assembly 702 is positioned on third RF coil assembly 802 and overlaps with RF coil assembly 302 at a second half (e.g., the right half) of the groin/pelvic region of patient 304. Accordingly, the first end of second RF coil assembly 702 is positioned on/proximate the first side of the groin, the second end of second RF coil assembly 702 is positioned on/proximate the second side of the groin, and the central section of second RF coil assembly 702 wraps around a curved/intersecting region of the groin (e.g., the right hip). Further, third RF coil assembly 802 may include a similar structure (e.g., similar number and/or arrangement of RF coils) relative to RF coil assembly 302 and second RF coil assembly 702.

The configurations shown in FIGS. 3-8 are exemplary, and other configurations are possible. For example, one or more RF coil assemblies as described herein may be used to image a foot/ankle, knee, wrist/arm, or other desired anatomical region. Further, while FIGS. 3-8 were described above as including RF coil assemblies similar to RF coil assembly 200, it should be appreciated that the RF coil assemblies described below with respect to FIGS. 9 and 10 may be worn in the same or similar configurations as shown in FIGS. 3-8.

FIGS. 9 and 10 show additional exemplary embodiments of bowtie RF coil assemblies that each include a higher density of RF coils than the RF coil assembly 200 of FIG. 2. FIG. 9 shows an RF coil assembly 900 that includes 38 total RF coils arranged similarly to the RF coils of RF coil assembly 200. RF coil assembly 900 may include several components similar to those described above with reference to RF coil assembly 200. Specifically, RF coil assembly 900 includes distal edge 902, distal edge 904, outer surface 995, interface board 985, coil-interfacing cable 950, connector 952, and port 948, similar to distal edge 202, distal edge 204, outer surface 295, interface board 285, coil-interfacing cable 250, connector 252, and port 248, respectively, described above with reference to RF coil assembly 200. Further, central longitudinal axis 954 and central transverse axis 956 of RF coil assembly 900 may be similar to central longitudinal axis 254 and central transverse axis 256, respectively, of RF coil assembly 200. The RF coil assembly 900 includes a plurality of flexible RF coils similar to the RF coils described below with reference to FIGS. 11A and 11B. One or more of the RF coils of the RF coil assembly 900 may be similar to the RF coils of the RF coil assembly 200. For example, an eccentricity of one or more of the RF coils of the RF coil assembly 900 may be similar to an eccentricity of one or more of the RF coils of the RF coil assembly 200 (e.g., similar to first RF coil 206, tenth RF coil 224, etc. shown by FIG. 2). Each of the RF coils of the RF coil assembly 900 includes coupling electronics (e.g., coupling electronics 938 of RF coil 906) similar to the coupling electronics 238 described above with reference to RF coil assembly 200. However, in FIG. 9, all other coupling electronics have been removed for clarity.

RF coil assembly 900 includes a first end 958 that extends along a first end length 958′, a second end 960 that extends along a second end length 960′, and a central section 980 extending between first end 958 and second end 960 and that extends along a central section length 980′. To form the bowtie shape, first end 958 narrows along central longitudinal axis 954 from distal edge 902 toward central transverse axis 956. Likewise, second end 960 narrows along central longitudinal axis 954 from distal edge 904 toward central transverse axis 956. Each of first side edge 944 and second side edge 946 slopes inward from distal edge 902 to central transverse axis 956 and slopes outward from central transverse axis 956 to distal edge 904, creating a most-narrow region at central transverse axis 956.

First end 958 includes 18 RF coils arranged into four overlapping rows. A first row of RF coils of first end 958 (closest to distal edge 902) includes six RF coils, a second row of RF coils of first end 958 includes five RF coils, a third row of RF coils of first end 958 includes four RF coils, and a fourth row of RF coils of first end 958 includes three RF coils. The RF coils of first end 958 may overlap in a similar manner to the RF coils of first end 258 of RF coil assembly 200.

Second end 960 includes 18 RF coils arranged into four overlapping rows. A first row of RF coils of second end 960 (closest to distal edge 904) includes six RF coils, a second row of RF coils of second end 960 includes five RF coils, a third row of RF coils of second end 960 includes four RF coils, and a fourth row of RF coils of second end 960 includes three RF coils. The RF coils of second end 960 may overlap in a similar manner to the RF coils of second end 260 of RF coil assembly 200.

Central section 980 includes two saddle RF coils, a first saddle RF coil 924 and a second saddle RF coil 925. Each of first saddle RF coil 924 and second saddle RF coil 925 is similar to tenth RF coil 224 of RF coil assembly 200, and thus each saddle RF coil is shaped as a figure-eight and is comprised of two overlapped/intersecting loops. First saddle RF coil 924 overlaps with two RF coils of the fourth row of RF coils of first end 958 and second saddle RF coil 925 overlaps with two RF coils of the fourth row of RF coils of first end 958. The middle RF coil of the fourth row of RF coils of first end 958 overlaps both first saddle RF coil 924 and second saddle RF coil 925. Likewise, first saddle RF coil 924 overlaps with two RF coils of the fourth row of RF coils of second end 960 and second saddle RF coil 925 overlaps with two RF coils of the fourth row of RF coils of second end 960. The middle RF coil of the fourth row of RF coils of second end 960 overlaps both first saddle RF coil 924 and second saddle RF coil 925.

First saddle RF coil 924 and second saddle RF coil 925 may have the same dimensions. The remaining RF coils (e.g., the circular RF coils of first end 958 and second end 960) may each have the same dimensions. For example, each circular RF coil of first end 958 and second end 960 may have a diameter of 9 or 10 cm, which may be smaller than the diameter of the circular RF coils of RF coil assembly 200 of FIG. 2. However, the dimensions provided herein are exemplary and other dimensions are possible without departing from the scope of this disclosure. Further, different RF coil assemblies may include different size RF coils based on the size of the patients to be imaged. Similar to RF coil assembly 200, central section 980 may be configured to bend or fold along central transverse axis 956. As such, first saddle RF coil 924 and second saddle RF coil 925 may be positioned with their respective intersecting regions aligned along central transverse axis 956 (or within a threshold distance of central transverse axis 956, such as within 1-2 cm of central transverse axis 956).

Each RF coil of RF coil assembly 900 includes a respective coupling electronics portion. For example, RF coil 906 includes coupling electronics portion 938, similar to first RF coil 206 and coupling electronics portion 238 of RF coil assembly 200. The remaining coupling electronics portions have been removed from FIG. 9 for clarity. Likewise, each coupling electronics portion is coupled to an output (e.g., a coil-interfacing cable or cable harness) that is electrically coupleable to the MRI system. For example, FIG. 9 shows coil-interfacing cable 950 having an output connector 952 adapted to couple to the MRI system in order to transmit electrical signals from the RF coils of the RF coil assembly 900 to the MRI system. Each RF coil may be electrically coupled with the coil-interfacing cable 950 and output connector 952 via respective coupling electronics. Specifically, the coupling electronics of each RF coil (e.g., the RF coils of the first end 958, second end 960, and central section 980) may be electrically coupled to interface board 985 via wires, and interface board 985 may be electrically coupled with output connector 952 via coil-interfacing cable 250. Each coupling electronics portion may be electrically coupled to the interface board 985 via one or more wires (not shown in FIG. 9 for clarity), and interface board 985 may transmit signals (e.g., electrical signals) from each coupling electronics portion to the output connector 952 via coil-interfacing cable 950. In some examples, the wires may be embedded within the material of the RF coil assembly 900, and may extend toward the interface board 985 in order to electrically couple the coupling electronics of each RF coil with the interface board 985.

Each RF coil, including first saddle RF coil 924 and second saddle RF coil 925, may have only one coupling electronics portion. In particular, while each saddle RF coil is comprised of two loops, the two loops are formed from a single loop that is twisted to form the saddle/figure-eight shape. Because each saddle RF coil is comprised of one loop that is twisted into the saddle shape, each saddle RF coil only includes one coupling electronics portion.

Coil-interfacing cable 950 may be electrically coupled to the interface board 985 via a port 948 (e.g., an opening). For example, coil-interfacing cable 950 may include a plurality of wires adapted to transmit electrical signals from the interface board 985 to the output connector 952. Coil-interfacing cable 950, interface board 985, port 948, and output connector 952 may be the same or similar to coil-interfacing cable 250, interface board 285, port 248, and output connector 252 of RF coil assembly 200, and thus description of coil-interfacing cable 250, interface board 285, port 248, and output connector 252 of RF coil assembly 200 provided above with respect to FIG. 2 likewise applies to coil-interfacing cable 950, interface board 985, port 948, and output connector 952 of RF coil assembly 900.

The port 948 and/or interface board 985 may be positioned at a suitable location on RF coil assembly 900. Accordingly, port 948, interface board 985, coil interfacing cable 950, and output connector 952 are shown in dashed lines in FIG. 9 in order to signify that port 948 and interface board 985 (and hence cable 950 and connector 952) may be positioned elsewhere on RF coil assembly 900 without departing from the scope of the disclosure.

Configuring the RF coil assembly 900 to include 38 RF coils may increase a signal to noise ratio of information obtained with RF coil assembly 900 relative to RF coil assemblies that include a lower number of RF coils (e.g., relative to RF coil assembly 200). Further, a higher number of RF coils in the RF coil assembly may increase acceleration factors for parallel imaging.

FIG. 10 shows an RF coil assembly 1000 that includes 63 total RF coils arranged similarly to the RF coils of RF coil assembly 200. RF coil assembly 1000 may include several components similar to those described above with reference to RF coil assembly 200. Specifically, RF coil assembly 1000 includes distal edge 1002, distal edge 1004, outer surface 1095, interface board 1085, coil-interfacing cable 1050, connector 1052, and port 1048, similar to distal edge 202, distal edge 204, outer surface 295, interface board 285, coil-interfacing cable 250, connector 252, and port 248, respectively, described above with reference to RF coil assembly 200. Further, central longitudinal axis 1054 and central transverse axis 1056 of RF coil assembly 1000 may be similar to central longitudinal axis 254 and central transverse axis 256, respectively, of RF coil assembly 200. The RF coil assembly 1000 includes a plurality of flexible RF coils similar to the RF coils described below with reference to FIGS. 11A and 11B. One or more of the RF coils of the RF coil assembly 1000 may be similar to the RF coils of the RF coil assembly 200. For example, an eccentricity of one or more of the RF coils of the RF coil assembly 1000 may be similar to an eccentricity of one or more of the RF coils of the RF coil assembly 200 (e.g., similar to first RF coil 206, tenth RF coil 224, etc. shown by FIG. 2). Each of the RF coils of the RF coil assembly 1000 includes coupling electronics (e.g., coupling electronics 1038 of RF coil 1006) similar to the coupling electronics 238 described above with reference to RF coil assembly 200. However, in FIG. 10, all other coupling electronics have been removed for clarity.

RF coil assembly 1000 includes a first end 1058 that extends along a first end length 1058′, a second end 1060 that extends along a second end length 1060′, and a central section 1080 extending between first end 1058 and second end 1060 and that extends along a central section length 1080′. To form the bowtie shape, first end 1058 narrows along central longitudinal axis 1054 from distal edge 1002 toward central transverse axis 1056. Likewise, second end 1060 narrows along central longitudinal axis 1054 from distal edge 1004 toward central transverse axis 1056. Each of first side edge 1044 and second side edge 1046 slopes inward from distal edge 1002 to central transverse axis 1056 and slopes outward from central transverse axis 1056 to distal edge 1004, creating a most-narrow region at central transverse axis 1056.

First end 1058 includes 30 RF coils arranged into five overlapping rows. A first row of RF coils of first end 1058 (closest to distal edge 1002) includes eight RF coils, a second row of RF coils of first end 1058 includes seven RF coils, a third row of RF coils of first end 1058 includes six RF coils, a fourth row of RF coils of first end 1058 includes five RF coils, and a fifth row of RF coils of first end 1058 includes four RF coils. The RF coils of first end 1058 may overlap in a similar manner to the RF coils of first end 258 of RF coil assembly 200.

Second end 1060 includes 30 RF coils arranged into five overlapping rows. A first row of RF coils of second end 1060 (closest to distal edge 1004) includes eight RF coils, a second row of RF coils of second end 1060 includes seven RF coils, a third row of RF coils of second end 1060 includes six RF coils, a fourth row of RF coils of second end 1060 includes five RF coils, and a fifth row of RF coils of second end 1060 includes four RF coils. The RF coils of second end 1060 may overlap in a similar manner to the RF coils of second end 260 of RF coil assembly 200.

Central section 1080 includes three saddle RF coils, a first saddle RF coil 924, a second saddle RF coil 925, and a third saddle RF coil 1026. Each of first saddle RF coil 1024, second saddle RF coil 1025, and third saddle RF coil 1026 is similar to tenth RF coil 224 of RF coil assembly 200, and thus each saddle RF coil is shaped as a figure-eight and is comprised of two overlapped/intersecting loops. First saddle RF coil 1024 overlaps with two RF coils of the fifth row of RF coils of first end 1058, second saddle RF coil 1025 overlaps with two RF coils of the fifth row of RF coils of first end 1058, and third saddle RF coil 1026 overlaps with two RF coils of the fifth row of RF coils of first end 1058. The middle two RF coils of the fifth row of RF coils of first end 1058 each overlaps two saddle RF coils. Likewise, first saddle RF coil 1024 overlaps with two RF coils of the fifth row of RF coils of second end 1060, second saddle RF coil 1025 overlaps with two RF coils of the fifth row of RF coils of second end 1060, and third saddle RF coil 1026 overlaps with two RF coils of the fifth row of RF coils of second end 1060. The middle two RF coils of the fifth row of RF coils of second end 1060 each overlaps two saddle RF coils.

First saddle RF coil 1024 and third saddle RF coil 1025 may have the same dimensions, while second saddle RF coil 1025 may different dimensions. In other examples, all three saddle RF coils may have the same dimensions. The remaining RF coils (e.g., the circular RF coils of first end 1058 and second end 1060) may each have the same dimensions. For example, each circular RF coil of first end 1058 and second end 1060 may have a diameter of 8 or 9 cm, which may be smaller than the diameter of the circular RF coils of RF coil assembly 200 of FIG. 2. However, the dimensions provided herein are non-limiting and other dimensions are possible. Further, the dimensions of the RF coils may depend on the size of the patient that is to be imaged. First saddle RF coil 1024, second saddle RF coil 1025, and third saddle RF coil 1026 may be positioned with their respective intersecting regions aligned along central transverse axis 1056 (or within a threshold distance of central transverse axis 1056, such as within 1-2 cm of central transverse axis 1056).

Each RF coil of RF coil assembly 1000 includes a respective coupling electronics portion. For example, RF coil 1006 includes coupling electronics portion 1038, similar to first RF coil 206 and coupling electronics portion 238 of RF coil assembly 200. The remaining coupling electronics portions have been removed from FIG. 10 for clarity. Likewise, each coupling electronics portion is coupled to an output (e.g., a coil-interfacing cable or cable harness) that is electrically coupleable to the MRI system. For example, FIG. 10 shows coil-interfacing cable 1050 having an output connector 1052 adapted to couple to the MRI system in order to transmit electrical signals from the RF coils of the RF coil assembly 1000 to the MRI system. Each RF coil may be electrically coupled with the coil-interfacing cable 1050 and output connector 1052 via respective coupling electronics. Specifically, the coupling electronics of each RF coil (e.g., the RF coils of the first end 1058, second end 1060, and central section 1080) may be electrically coupled to interface board 1085 via wires, and interface board 1085 may be electrically coupled with output connector 1052 via coil-interfacing cable 1050. Each coupling electronics portion may be electrically coupled to the interface board 1085 via one or more wires (not shown in FIG. 10 for clarity), and interface board 1085 may transmit signals (e.g., electrical signals) from each coupling electronics portion to the output connector 1052 via coil-interfacing cable 1050. In some examples, the wires may be embedded within the material of the RF coil assembly 1000, and may extend toward the interface board 1085 in order to electrically couple the coupling electronics of each RF coil with the interface board 1085.

Each RF coil, including first saddle RF coil 1024, second saddle RF coil 1025, and third saddle RF coil 1026, may have only one coupling electronics portion. In particular, while each saddle RF coil is comprised of two loops, the two loops are formed from a single loop that is twisted to form the saddle/figure-eight shape. Because each saddle RF coil is comprised of one loop that is twisted into the saddle shape, each saddle RF coil only includes one coupling electronics portion.

Coil-interfacing cable 1050 may be electrically coupled to the interface board 1085 via a port 1048 (e.g., an opening). For example, coil-interfacing cable 1050 may include a plurality of wires adapted to transmit electrical signals from the interface board 1085 to the output connector 1052. Coil-interfacing cable 1050, interface board 1085, port 1048, and output connector 1052 may be the same or similar to coil-interfacing cable 250, interface board 285, port 248, and output connector 252 of RF coil assembly 200, and thus description of coil-interfacing cable 250, interface board 285, port 248, and output connector 252 of RF coil assembly 200 provided above with respect to FIG. 2 likewise applies to coil-interfacing cable 1050, interface board 1085, port 1048, and output connector 1052 of RF coil assembly 1000.

The port 1048 and/or interface board 1085 may be positioned at a suitable location on RF coil assembly 1000. Accordingly, port 1048, interface board 1085, coil interfacing cable 1050, and output connector 1052 are shown in dashed lines in FIG. 10 in order to signify that port 1048 and interface board 1085 (and hence cable 1050 and connector 1052) may be positioned elsewhere on RF coil assembly 1000 without departing from the scope of the disclosure.

Configuring the RF coil assembly 1000 to include 63 RF coils may increase a signal to noise ratio of information obtained with RF coil assembly 1000 relative to RF coil assemblies that include a lower number of RF coils (e.g., relative to RF coil assembly 200 or RF coil assembly 900). Further, a higher number of RF coils in the RF coil assembly may increase acceleration factors for parallel imaging.

While each of the RF coil assemblies described above with respect to FIGS. 2, 9, and 10 include substrate material (such as the outer surfaces described above) shaped as symmetric flaps that join at a central transverse axis, other shapes are possible. For example, rather than gradually narrowing in width from a respective distal edge until the central transverse axis, each flap/end may narrow in width from a distal edge until a point spaced away from the central transverse axis (e.g., 3-5 cm from the central transverse axis). In such examples, the central section may include a rectangular section of material between the flaps/ends, or the central section may narrow at a different angle to the central transverse axis. Other shapes are possible without departing from the scope of this disclosure.

Turning now to FIG. 11A, a schematic view of an RF coil 1102 coupled to a controller unit 1110 is shown according to an exemplary embodiment. The RF coil 1102 includes a circular loop portion 1101 and a coupling electronics portion 1103 which is coupled to the controller unit 1110 via a coil-interfacing cable 1112. In some embodiments, the RF coil may be a surface receive coil, which may be single- or multi-channel. The RF coil 1102 may be used in RF coil unit 14 of FIG. 1 and as such may operate at one or more frequencies in the MRI apparatus 10. RF coil 1102 is a non-limiting example of circular RF coils that may be included in the RF coil assemblies of FIGS. 2, 9, and/or 10. The coil-interfacing cable 1112 may extend between the coupling electronics portion 1103 and an interfacing connector of an RF coil array and/or between the interfacing connector of the RF coil array and the MRI system controller unit 1110. The controller unit 1110 may correspond to and/or be associated with the data processing unit 31 and/or controller unit 25 in FIG. 1.

The loop portion 1101 may be comprised of at least two parallel conductors that form a distributed capacitance along the length of the loop portion. In the example shown in FIG. 11A, the loop portion 1101 includes a first conductor 1120 and a second conductor 1122 which exhibit a substantially uniform capacitance along the entire length of the loop portion. Distributed capacitance (DCAP), as used herein, represents a capacitance exhibited between conductors that distributes along the length of the conductors and may be void of discrete or lumped capacitive components and discrete or lumped inductive components. The DCAP can also be called incorporated capacitance. In some embodiments, the capacitance may distribute in a uniform manner along the length of the conductors.

A dielectric material 1124 encapsulates and separates the first and second conductors 1120, 1122. The dielectric material 1124 may be selected to achieve a desired distributive capacitance. For example, the dielectric material 1124 may be selected based on a desired permittivity E. In particular, the dielectric material 1124 may be air, rubber, plastic, or any other appropriate dielectric material. In some embodiments, the dielectric material may be polytetrafluoroethylene (pTFE). The dielectric material 1124 may surround the parallel conductive elements of the first and second conductors 1120, 1122. Alternatively, the first and second conductors 1120, 1122 may be twisted upon one another to from a twisted pair cable. As another example, the dielectric material 1124 may be a plastic material. The first and second conductors 1120, 1122 may form a coaxial structure in which the plastic dielectric material 1124 separates the first and second conductors. As another example, the first and second conductors may be configured as planar strips.

While FIG. 11A includes the loop portion being circular, other shapes are possible, such as oval or rectangular. However, the loop portion of RF coil 1102 is planar and does not overlap or twist on itself.

The coupling electronics portion 1103 is connected to the loop portion 1101 of the RF coil 1102. Herein, the coupling electronics portion 1103 may include a decoupling circuit 1104, impedance inverter circuit 1106, and a pre-amplifier 1108. The decoupling circuit 1104 may effectively decouple the RF coil during a transmit operation. Typically, the RF coil 1102 in its receive mode may receive MR signals from a body of a subject being imaged by the MR apparatus. If the RF coil 1102 is not used for transmission, then it may be decoupled from the RF body coil while the RF body coil is transmitting the RF signal.

The impedance inverter circuit 1106 may include an impedance matching network between the loop portion 1101 and the pre-amplifier 1108. The impedance inverter circuit 1106 is configured to transform an impedance of the loop portion 1101 into an optimal source impedance for the pre-amplifier 1108. The impedance inverter circuit 1106 may include an impedance matching network and an input balun. The pre-amplifier 1108 receives MR signals from the loop portion 1101 and amplifies the received MR signals. In one example, the pre-amplifier 1108 may have a low input impedance configured to accommodate a relatively high blocking or source impedance. The coupling electronics portion 1103 may be packaged in a very small PCB, e.g., approximately 2 cm² in size or smaller. The PCB may be protected with a conformal coating or an encapsulating resin.

The coil-interfacing cable 1112, such as a RF coil array interfacing cable, may be used to transmit signals between the RF coils and other aspects of the processing system. The RF coil array interfacing cable may be disposed within the bore or imaging space of the MRI apparatus (such as MRI apparatus 10 of FIG. 1) and subjected to electro-magnetic fields produced and used by the MRI apparatus. In MRI systems, coil-interfacing cables, such as coil-interfacing cable 1112, may support transmitter-driven common-mode currents, which may in turn create field distortions and/or unpredictable heating of components. Typically, common-mode currents are blocked by using baluns. Baluns or common-mode traps provide high common-mode impedances, which in turn reduces the effect of transmitter-driven currents. Thus, coil-interfacing cable 1112 may include one or more baluns. In some embodiments, the one or more baluns may be continuous baluns, such as distributed, flutter, and/or butterfly baluns. The cable 1112 may be a 3-conductor triaxial cable having a center conductor, an inner shield, and an outer shield. In some embodiments, the center conductor is connected to the RF signal and pre-amp control (RF), the inner shield is connected to ground (GND), and the outer shield is connected to the multi-control bias (diode decoupling control) (MC_BIAS).

FIG. 11B shows a schematic view of an RF coil 1152 according to an exemplary embodiment. In some embodiments, the RF coil may be a surface receive coil, which may be single- or multi-channel. The RF coil 1152 may be used in RF coil unit 14 of FIG. 1 and as such may operate at one or more frequencies in the MRI apparatus 10. The RF coil 1152 includes a saddle shaped loop portion 1151. RF coil 1102 is a non-limiting example of saddle RF coils that may be included in the RF coil assemblies of FIGS. 2, 9, and/or 10. RF coil 1152 includes coupling electronics portion 1103 which is coupled to the controller unit 1110 via a coil-interfacing cable 1112, similar to RF coil 1102 of FIG. 11A.

The loop portion 1151 may be comprised of at least two parallel conductors that form a distributed capacitance along the length of the loop portion. In the example shown in FIG. 11B, the loop portion 1151 includes a first conductor 1160 and a second conductor 1162 which exhibit a substantially uniform capacitance along the entire length of the loop portion. A dielectric material 1164 encapsulates and separates the first and second conductors 1160, 1162. The two conductors and dielectric material may be configured similarly to first and second conductors 1120, 1122 and dielectric material 1124 of FIG. 11A, and thus the description of first and second conductors 1120, 1122 and dielectric material 1124 likewise applies to first and second conductors 1160, 1162 and dielectric material 1164.

The first and second conductors 1160, 1162 and dielectric material 1164 are twisted into the saddle/figure-eight shape. As appreciated by FIG. 11B, first conductor 1160 may be an outer conductor on a first side of the loop portion and may switch to being an inner conductor at intersecting region 1166. Likewise, second conductor 1162 may be an inner conductor and may switch at intersecting region 1166 to being an outer conductor. At the intersecting region 1166, the conductors and dielectric material may twist such that a first segment of the conductors and dielectric material is positioned on top of a second segment of the conductors and dielectric material.

The RF coils presented above with respect to FIGS. 11A and 11B may be utilized in order to receive MR signals during an MR imaging session. As such, the RF coils of FIGS. 11A and 11B may be used in RF coil unit 14 of FIG. 1 and may be coupled to a downstream component of the MRI system, such as the controller unit 25. The RF coils may be placed in the bore of the MRI system in order to receive the MR signals during the imaging session, and thus may be in proximity to the transmit RF coil (e.g., the body RF coil unit 15 of FIG. 1). The controller unit may store instructions in non-transitory memory that are executable to generate an image from an imaging subject positioned in the bore of the MRI system during an MR imaging session. To generate the image, the controller unit may store instructions to perform a transmit phase of the MR imaging session. During the transmit phase, the controller unit may command (e.g., send signals) to activate the transmit RF coil(s) in order to transmit one or more RF pulses. To prevent interference leading to B₁ field distortion during the transmit phase, the receive RF coil(s) may be decoupled during the transmit phase. The controller unit may store instructions executable to perform a subsequent receive phase of the MR imaging session. During the receive phase, the controller unit may obtain MR signals from the receive RF coil(s). The MR signals are usable to reconstruct the image of the imaging subject positioned in the bore of the MM system.

FIGS. 2, 9, and 10 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

The technical effect of configuring the RF coil assembly to include a first end having a first RF coil set of circular RF coils, a second end having a second RF coil set of circular RF coils, and a central section joined to the first end and second end and having a third RF coil set of saddle RF coils, is to enable the RF coil assembly to image through and/or around curved anatomical features without signal loss.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A radio frequency (RF) coil assembly for a magnetic resonance imaging (MRI) system, comprising:
a first end including a first set of flexible RF coil elements having a first shape;
a second end including a second set of flexible RF coil elements having the first shape;
a central section extending between the first end and the second end and including a flexible, saddle shaped RF coil element, the first end and the second end bendable to the central section, the saddle shaped RF coil element having a different shape than the first shape; and
where the saddle shaped RF coil element and each RF coil element of the first set of RF coil elements and the second set of RF coil elements includes a coupling electronics portion and at least two parallel, distributed capacitance wire conductors encapsulated and separated by a dielectric material.

2. The RF coil assembly of claim 1, wherein the first shape is circular and wherein the saddle shaped RF coil element is a twisted loop.

3. The RF coil assembly of claim 2, wherein the RF coil assembly has an axis of symmetry at a central transverse axis that bisects the central section, and wherein a twist of the twisted loop of the saddle shaped RF coil is aligned along the central transverse axis.

4. The RF coil assembly of claim 3, wherein the first end and the second end each narrow from a respective distal edge toward the central transverse axis and wherein the central section includes a most-narrow region of the RF coil assembly.

5. The RF coil assembly of claim 1, wherein the saddle shaped RF coil element and each RF coil element of the first set of RF coil elements and the second set of RF coil elements overlaps at least two other RF coil elements.

6. The RF coil assembly of claim 1, further comprising a coil-interfacing cable extending outward from a port of the RF coil assembly, wherein the coil-interfacing cable is electrically connected to the saddle shaped RF coil, the first set of RF coils, and the second set of RF coils.

7. The RF coil assembly of claim 1, wherein the first set of RF coil elements and the second set of RF coil elements each include nine RF coil elements.

8. The RF coil assembly of claim 7, wherein the nine RF coil elements of the first set of RF coil elements and the nine RF coil elements of the second set of RF coil elements are each arranged in three respective rows, with a first row including four RF coil elements, a second row including three RF coil elements, and a third row including two RF coil elements.

9. The RF coil assembly of claim 1, wherein the first set of RF coil elements and the second set of RF coil elements each include eighteen RF coil elements, wherein the saddle shaped RF coil element is a first saddle shaped RF coil element, and wherein the central section further includes a second saddle shaped RF coil element.

10. The RF coil assembly of claim 9, wherein the eighteen RF coil elements of the first set of RF coil elements and the eighteen RF coil elements of the second set of RF coil elements are each arranged in four respective rows, with a first row including six RF coil elements, a second row including five RF coil elements, a third row including four RF coil elements, and a fourth row including three RF coil elements.

11. The RF coil assembly of claim 1, wherein the first set of RF coil elements and the second set of RF coil elements each include thirty RF coil elements, wherein the saddle shaped RF coil element is a first saddle shaped RF coil element, and wherein the central section further includes a second saddle shaped RF coil element and a third saddle shaped RF coil element.

12. The RF coil assembly of claim 11, wherein the thirty RF coil elements of the first set of RF coil elements and the thirty RF coil elements of the second set of RF coil elements are each arranged in five respective rows, with a first row including eight RF coil elements, a second row including seven RF coil elements, a third row including six RF coil elements, a fourth row including five RF coil elements, and a fifth row including four RF coil elements.

13. A wearable radio frequency (RF) coil assembly for a magnetic resonance imaging (MRI) system, comprising:
a body configured to be worn by a subject being scanned, the body comprising:
a first end including a first set of flexible, circular shaped RF coils, wherein the first end is configured to wrap around a first side of the subject;
a second end including a second set of flexible, circular shaped RF coils, wherein the second end is configured to wrap around a second side of the subject; and
a central section extending between the first end and the second end and including at least one flexible, saddle shaped RF coil element,
wherein each RF coil element of the first end, the second end, and the central section includes a coupling electronics portion and at least two parallel, distributed capacitance wire conductors encapsulated and separated by a dielectric material.

14. The wearable RF coil assembly of claim 13, wherein the first set of circular shaped RF coils consists of nine RF coil elements, the second set of circular shaped RF coils consists of nine RF coil elements, and the central section includes only one saddle shaped RF coil element.

15. The wearable RF coil assembly of claim 13, wherein the first set of circular shaped RF coils consists of eighteen RF coil elements, the second set of circular shaped RF coils consists of eighteen RF coil elements, and the at least one saddle shaped RF coil element consists of two saddle shaped RF coil elements.

16. The wearable RF coil assembly of claim 13, wherein the first set of circular shaped RF coils consists of thirty RF coil elements, the second set of circular shaped RF coils consists of thirty RF coil elements, and the at least one saddle shaped RF coil consists of three saddle shaped RF coil elements.

17. The wearable RF coil assembly of claim 13, wherein the body is formed of a flexible material transparent to RF signals, and the first and second sets of circular shaped RF coils and the at least one saddle shaped RF coil element are embedded within the flexible material.

18. A radio frequency (RF) coil assembly for a magnetic resonance imaging (MRI) system, comprising:
a first end including a first set of flexible RF coil elements having a first shape;
a second end including a second set of flexible RF coil elements having the first shape;
a central section extending between the first end and the second end and including a flexible, saddle shaped RF coil element; and
where the saddle shaped RF coil element and each RF coil element of the first set of RF coil elements and the second set of RF coil elements includes a coupling electronics portion and at least two parallel, distributed capacitance wire conductors encapsulated and separated by a dielectric material,
where the RF coil assembly includes a first axis of symmetry that bisects the central section and the saddle shaped RF coil element, the first end and the second end bendable to the central section at the first axis of symmetry.

19. The RF coil assembly of claim 18, wherein the saddle shaped RF coil element includes a twisted loop having an intersecting region, wherein the first axis of symmetry bisects the intersecting region, and wherein the first shape is circular.

20. The RF coil assembly of claim 18, wherein the RF coil assembly includes a second axis of symmetry that bisects the first end, the second end, and the central section.
